(19) United States
(12) Patent Application Publication
(10) Pub. No.: US 2021/0038156 A1
(43) Pub. Date: Feb. 11, 2021

(54) STABILIZING ACCESSORY FOR ADHESIVE MEDICAL DEVICES

(71) Applicant: West Affum Holdings Corp., Grand Cayman (KY)

(72) Inventors: Jaeho Kim, Kirkland, WA (US); Kelly A. Brennan, Kirkland, WA (US)

(21) Appl. No.: 16/921,757

(22) Filed: Jul. 6, 2020

Related U.S. Application Data
(60) Provisional application No. 62/883,544, filed on Aug. 6, 2019.

(51) Int. Cl. A61B 5/00; A61B 5/0408; D02G 3/32; D02G 3/04; D02G 3/44

(52) U.S. Cl. CPC A61B 5/6831; A61B 5/6832; A61B 5/0408; D02G 3/448; D02G 3/328; D02G 3/045; A61B 5/0022

(57) ABSTRACT

Technologies and implementations for stabilizing a wearable medical device (WMD) on a person. The technologies and implementations facilitate improved stability of adhesion and contact of the WMD on the person. Additionally, the technologies and implementations include length changeable support structure configured to help facilitate the improved stability.

### RELATED APPLICATION

This application claims benefit of priority to U.S. Provisional Patent Application Ser. No. 62/883,544, filed on Aug. 6, 2019, titled STRAP ACCESSORY FOR AN ADHESIVE WEARABLE MEDICAL DEVICE, which is incorporated herein by reference in its entirety.

### INFORMATION

Technology has contributed to improvements in healthcare. Some examples include healthcare related devices that may be mobile and personal. Mobile and personal healthcare devices may include Wearable Medical Devices (WMDs). Some WMDs may include medical devices that facilitate monitoring of various health related activities of a person. For example, a WMD may include a medical device that may be used to monitor a person's heart activity. The heart activity monitored by the WMD may be in the form of electrical signals (i.e., electrocardiogram or ECG). The WMD may be in a form factor capable of being worn by a person, who's heart activity is to be monitored. Monitoring of a person's ECG may facilitate intervention of heart related issues.

An example of a WMD that may be used to monitor and facilitate intervention of a person's heart activity may be a cardioverter defibrillator type medical device (i.e., wearable cardioverter defibrillator or WCD). In order to help facilitate monitoring and intervention, a WCD may be configured to adhere to a person's body (i.e., adhere to the skin of the person).

In an example of WCD, a WCD may include various components to facilitate monitoring and treating of a person's heart (the wearer). In order to help facilitate monitoring of the person's heart, the WCD may be attachable on the person. The WCD may be attachable on the person by adhesive techniques. For example, the WCD may have an adhesive surface to attach on a surface of the skin of the person (e.g., location around the waist). Additionally, the WCD may be coupled to an electrode that may be attached to another surface of the skin of the person (e.g., location around the person's heart). How well the WCD is attached to the person may be of consideration in the monitoring and treating of the person (e.g., the adhesion of the WCD on the person's skin).

Maintaining attachment of a WCD on a person may be have its challenges because a common method of attachment may be on the skin of the person. The skin of a person may vary from person to person. For example, some people may have skin (e.g., in the areas where the WCD may be attached), that may be covered with body hair, prone to sweating, loose or excessive, dry or moist, etc. Additionally, movement of the person may affect the attachment of the WCD on the person. For example, movements such as “jiggling”, rippling, folding, rolling, and undulating movement of skin underneath the adhesion may affect the attachment of the WCD (e.g., separation of the WCD from the skin of the person). Ordinary movements such as rolling over in a lying position, sitting up, bending over, sitting down, coughing, sneezing, etc. may affect the adhesion of the WCD on the person's skin. Further, the weight, formfactor, and bulkiness of the WCD may affect the adhesion of the WCD on the person's skin. The above described factors and others that may affect adhesion of the WCD to the person can be referred to herein as adhesion factors. Accordingly, maintaining attachment of a WMD on a person may be affected by various considerations including adhesion factors.

The foregoing summary is illustrative only and not intended to be in any way limiting. In addition to the illustrative aspects, embodiments, and features described above, further aspects, embodiments, and features will become apparent by reference to the drawings and the following detailed description.

### SUMMARY

Described herein are various illustrative systems for improved attachment of a wearable medical device (WMD). Example systems may include a WMD and a support structure. The WMD may be configured to have one or more components that are adhesively attachable to a person. The support structure may be configured to couple with the WMD. Additionally, the support structure may have a length or dimension that is changeable (also referred to herein as length changeable) to facilitate attachment and/or maintenance of attachment of the WMD to the person.

The present disclosure also describes an apparatus for improved attachment of a wearable medical device (WMD). The apparatus may include a support structure. The support structure may be configured to couple with a WMD and be length changeable. The apparatus may include a length changeable control module communicatively coupled to the support structure. The length changeable control module may be configured to change a length or dimension of the support structure responsive to a received signal indicating an attachment issue from the WMD.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates a system for consistent attachment of a wearable medical device (WMD) in accordance with various embodiments;

FIG. 2 illustrates a system for consistent attachment of a wearable medical device (WMD) in accordance with various alternative embodiments; and

FIG. 3 illustrates utilization of a system for consistent attachment of a wearable medical device (WMD) in accordance with various alternative embodiments.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to apparatus, and systems related to a providing an improved attachment of a wearable medical device (WMD).

Wearable medical devices (WMD) may be used to facilitate monitoring and treatment of various medical conditions of a person. In order to facilitate monitoring and treatment of medical conditions of a person, a WMD may be attachable on the person. A WMD that may be attachable to a person to help facilitate monitoring and treatment of a person may include a WMD configured to facilitate monitoring and treatment of potential issues with a person's heart. For example, a person may have a health condition, where the person may require a defibrillator (i.e., Arrhythmia). The defibrillator may be in the form of a cardioverter defibrillator (e.g., wearable cardioverter defibrillator or WCD), which may help facilitate prevention of Sudden Cardiac Death (SCD). The WCD may be attachable on the person using adhesive methods, where the WCD may include a WCD module and/or one or more electrodes. The WCD module may be attached to the body proximate the waist of the person, while the one or more electrodes may be attached proximate to the heart of the person. If the person experiences a heart condition requiring treatment, the WCD module may facilitate detection of the heartbeat issue, and responsive to the detected heart best issue, the WCD module may facilitate treatment of the person's heart via the one or more electrodes.

Before turning to the figures, a non-limiting example application of the various embodiments of the present disclosure is described. In the non-limiting example, a wearable medical device (WMD) may be utilized to facilitate monitoring and treatment of a person. An example of a WMD to facilitate monitoring and treatment of a person may be a WMD to monitor and treat a person's heart such as, but not limited to, a wearable cardioverter defibrillator (WCD). In one example, the WCD may be made up of two parts, a WCD or an electronics module having majority of the electronic components to facilitate monitoring and/or treatment of the heart and one or more electrodes to facilitate reading of the activities of the heart and administration of the treatment (e.g., an electric shock for defibrillation, cardioversion and/or pacing). In order to describe the disclosed subject matter, this example WCD may have a single electrode. However, it is understood that the claimed subject matter may include one or more electrodes. Further, the one or more electrodes may be different combinations of adhesive and non-adhesive electrodes in various embodiments. Still further, the one or more electrodes may be part of the electronics module in some embodiments, while in other embodiments the one or more other electrodes various combinations of electrodes that are separate from and/or part of the electronics module.

Each of the parts of the WCD may include an attachable surface such as, but not limited to, an adhesive surface. For example, the electrode may have an adhesive surface to attach the electrode on the skin near the person's heart. The electrode may help facilitate monitoring of the electrical activity of the person's heart. Commonly, the electrode may comprise of a relatively thin membrane having an electrical contact. On the other hand, WCD module may include the various electronic components to facilitate monitoring and treatment of the person's heart (e.g., power supply) resulting in some added bulk and weight as compared to the weight of the electrode. In some embodiments, the WCD module may have an adhesive surface to attach on the skin on some other part of the person's body such as, but not limited to, the person's waist. The WCD module and the electrode may have a cable coupling each other. Together, the WCD module and the electrode may be attached to the body of the person via the adhesive surfaces.

In accordance with the present disclosure, a support structure may be coupled to the WCD module attached around the waist. The support structure may help facilitate consistent attachment of the WCD module. As used herein, consistent attachment includes attachment so that the WCD module is in contact with the patient's skin sufficiently for accurate physiological signal sensing and/or effective therapy delivery, despite one or more of the aforementioned adhesion factors. In one example, the support structure may be in the form of a strap structured for fitting to the WCD module. For example, the strap may include a portion sized or configured to be positioned over the WCD module to help hold the WCD module against the patient's skin when the WCD module and support structure are being worn by the patient. In some embodiments, the strap portion may include a stretchable or elastic material. The strap may be attachable to the WCD module and its length may be changeable (e.g., a belt like strap having a buckle to facilitate adjustment of the strap). Accordingly, the strap may be adjusted to tighten around the waist of the person to firmly hold the WCD attached to the skin of the person (i.e., facilitate consistent attachment of the WCD on the person). In another example, the support structure may be in the form of a mesh. The mesh may be configured to wrap around the WCD (i.e., one or more of the WCD module or separate electrodes) and around the waist of the person. As will be described in detail, the mesh may be length changeable in a variety of manners to facilitate consistent attachment of the WCD on the person (e.g., made of stretchy, elastic, and/or resilient material, stimuli modifiable material, and the like). In a further example, the support structure may be controllable by mechanical and/or electrical stimuli responsive to a sensor included in the WCD or external to the WCD. Accordingly, the WCD may be consistently attached to the person in accordance with the various embodiments disclosed.

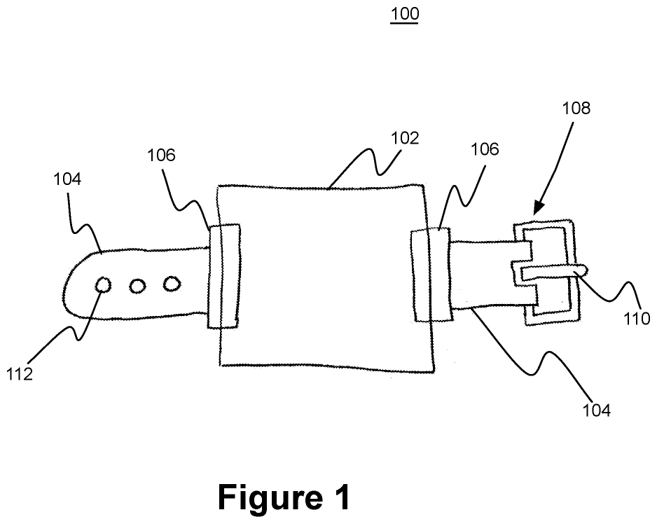

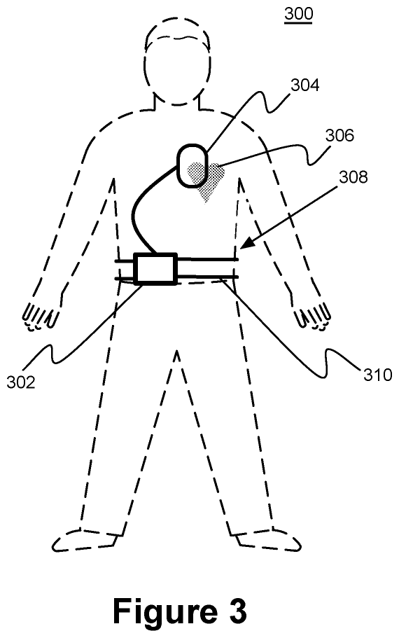

Turning now to FIG. 1, FIG. 1 illustrates a system for consistent attachment of a wearable medical device (WMD) in accordance with various embodiments. In FIG. 1, a system 100 may include a WMD 102 and a support structure 104. Additionally, the system 100 may include a coupling mechanism 106 configured to facilitate coupling of the support structure 104 with the WMD 102. Shown in FIG. 1, the support structure 104 may include a length changeable methodology 108. The length changeable methodology 108 may comprise of a center bar buckle 110 on one end of the support structure 104 and holes 112 on another end of the support structure 104. The center bar buckle 110 and the holes 112 may be used together to tighten the support structure 104 around a person 300 (shown in FIG. 3) (e.g., around the waist of the person 300). Accordingly, the system 100 may facilitate consistent attachment of the WMD 102 in accordance with various embodiments of the present disclosure.

In FIG. 1, the WMD 102 may be a wide variety of WMDs that may be attachable on a person such as, but not limited to, wearable biosensor, wearable cardioverter defibrillator (WCD), etc. For example, a wearable biosensor may include a wearable biosensor available from Koninklijke Philips N.V. of Amsterdam, Netherlands and a WCD may include a WCD available from Element Science, Inc. of San Francisco, Calif. The changeable methodology 108 of the support structure 104 may be shown as comprising the center bar buckle 110 with holes 112. However, the changeable methodology 108 may comprise a variety of changeable methodologies such as, but not limited to, cam lock, D-ring, T-lock, etc. with some examples being available from Bison Designs, LLS of Longmont, Colo., and accordingly, the claimed subject matter is not limited in this respect.

In some examples, the changeable methodology 108 may be in the form of a module (similar to some examples described later). For example, the changeable methodology 108 may not have the center bar buckle 110 with holes 112, but instead, the ends of the support structure 104 may be coupled at the module (i.e., a strap around the waist meeting at the module). In this example, the module may include mechanical components such as, but not limited to pins and rollers. The pins and rollers may be controlled by mechanical gears, where these gears may be actuated either by a small motor or manually. Actuating the pins and rollers may facilitate consistent attachment of the WMD 102 (e.g., tighten and/or loosen the support structure 104). Additionally, the actuation may be facilitated responsive to some form of stimulus as will be described below.

In FIG. 1, the coupling mechanism 106 may be a variety of coupling mechanisms such as, but not limited to, loops (e.g., belt loop type), hook-and-loop/ hook-and-pile fasteners (e.g., Velcro® available from Velcro Ltd. of Cheshire, United Kingdom), pin and slot type fasteners (e.g., Apple Watch band fastener method available from Apple Inc. of Cupertino, Calif.), etc. and accordingly, the claimed subject matter is not limited in this respect. Additionally, the support structure 104 may be made of a variety of material such as, but not limited to leather, nylon (e.g., nylon webbing), cotton, a combination thereof, etc. Accordingly, similar to some examples shown below, the coupling mechanism 106 may be of a form factor configured to cover the WMD 102 (i.e., the support structure 104 may encompass the WMD 102 going around the person 300), the claimed subject matter is not limited in this respect.

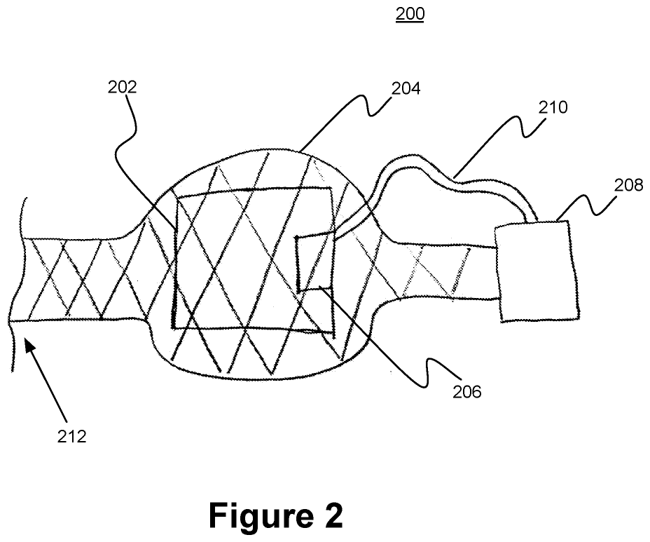

FIG. 2 illustrates a system for consistent attachment of a wearable medical device (WMD) in accordance with various alternative embodiments. Shown in FIG. 2, a system 200 may comprise of a WMD 202 and a support structure 204. In accordance with some embodiments, the WMD 202 in FIG. 2 may include an electronic control module 206. Correspondingly, the support structure 204 may have a length changeable control module 208. The electronic control module 206 of the WMD 202 may be communicatively coupled to the length changeable control module 208 of the support structure 204 via an electronic link 210. In FIG. 2, the support structure 204 may be in the form of a mesh configured to wrap around the WMD 202. Additionally, the support structure 204 may be configured to wrap around the body of a person (e.g., around the waist) having an end 212 coupled to the length changeable control module 208.

In the example of FIG. 2, the WMD 202 may be the WCD 102 as previously described. The electronic control module 206 may be configured to detect potential issues with the consistency of attachment of the WMD and compensate for the issues. For example, the electronic control module 206 may detect that the adhesion of the WMD on the skin may be separating and/or separated. The electronic control module 206 may communicate a signal to the length changeable control module 208 to change the length of the support structure 204 to compensate for the adhesion issue (i.e., shorten the length of the support structure 204 to tighten the support structure around the body, thereby pressing the WMD 202 against the skin of the body). Once the support structure 204 has tightened enough to restore a good adhesion of the WMD 202 on the skin, the electronic control module 206 may communicate a signal to the length changeable control module 208 to cease the change in length of the support structure 204. Since the communication between the electronic control module 206 and the length changeable control module 208 may be executed as necessary, the system 200 may facilitate consistent attachment of the WMD 202 in accordance with various embodiments of the present disclosure. The power to facilitate this type of adjustments may be provided by the WMD 204 and/or the length changeable control module 208 (e.g., a power supply included in the length changeable control module 208).

In FIG. 2, the support structure 204 may be of a mesh structure. However, the support structure 204 may be a variety of structure and material as is contemplated within the scope of the present disclosure. For example, the support structure 204 may be made of stretchable material such as, but not limited to, elastane, spandex, nylon, microfiber, cotton, and combinations thereof. The supports structure 204 being of a stretchable material may facilitate consistent attachment of the WMD 202 with the length changeable control module 208 being optional. For example, the support structure 204 may be of a spandex material having a fine mesh structure (e.g., pantyhose structure) providing an elastic body-hugging support structure holding the WMD 202 relatively tightly against the skin. In this example, the support structure 204 may not include the length changeable control module 208, which in turn the WMD 202 may not include the electronic control module 206. In addition to the support structure 204 being of a stretchable material, it is contemplated within the scope of the claimed subject matter that the material of the support structure may include material configured to provide comfort such as, but not limited to, breathable material (e.g., moisture wicking material). For example, the support structure 204 may be in the form of a spandex tube-like structure. The tube-like structure may be configured as to be pulled over the body and tighten around the body (“hug”), while covering the WMD 202. Additionally, the spandex tube may be configured to be changeable as may be described below.

In another example, the support structure 204 of FIG. 2 may be made of smart textile (can also be referred to as a smart fabric). Smart textile may be material configured to react to stimuli from various sources such as, but not limited to, mechanical, thermal chemical, electrical, magnetic sources, or any combination thereof. For example, the support structure 204 may be of a smart textile configured to react to electrical sources. In this example, the support structure 204 may not include the length changeable control module 208 and be of a mesh structure wrapping around the WMD 202 and around the body of the person 300. The support structure 204 may react to an electrical impulse from a power source such as may be included in the WMD 202 (not shown). The electrical impulse may be provided by an electronic link similar to the electronic link 210 shown or by contactless power transfer such as, but not limited to, inductive coupling. In this example, the WMD 202 may detect an issue with the attachment (e.g., adhesion) of the WMD 202 on the skin, and responsive to the detected issue, send an electrical signal to the support structure 204, which is made of smart textile, which in turn would cause the support structure 204 to react (e.g., tighten the mesh around the body or tighten the tube around the body). As previously alluded, the length changeable control module 208 may include mechanical components such as, but not limited to pins and rollers. The pins and rollers may be controlled by mechanical gears, where these gears may be actuated either by a small motor or manually. Responsive to stimuli from various sources, the pins and rollers may be actuated to facilitate consistent attachment of the WMD 202 (e.g., tighten and/or loosen the support structure 204). Accordingly, the support structure 204 facilitates consistent attachment of the WMD 202 in accordance with various embodiments of the present disclosure.

Continuing to refer to FIG. 2, it should be pointed out that even though the electronic control module 206 may be shown as being included in the WMD 202. However, the electronic control module 206 may be external to the WMD 202. Accordingly, in one example, the electronic control module 206 may be included in the length changeable control module 208. In this example, the length changeable control module 208 may have an accelerometer, which can detect motion of the person 300. The length changeable control module 208 may detect a change in motion or motion, which may affect the attachment of the WMD 202 on the body (e.g., running, falling, moving from a rest position, etc.). Responsive to the detected motion, the electronic control module 206 may transmit a signal (connective or contactless) to the length changeable control module 208, thereby causing the support structure 204 to react (e.g., tighten). It should be pointed out that even though some of the described examples may have been situations where the support structure 204 may react by tightening around the body to help facilitate attachment (e.g., adhesion) of the WMD 202, there may be situations where the support structure 204 may react by loosening around the body, and accordingly, the claimed subject matter is not limited in this respect.

In other examples, the electronic control module 206, may be in the form as an application configured to communicate with the length changeable control module 208. As shown in FIG. 2, the communication may be in the form of an electronic link 210. However, it is contemplated within the scope of the disclosure that the electronic link 210 may include wireless communication links such as, but not limited to, near field communication (NFC), Bluetooth®, Internet of Things (IoT) communication standards/protocols (e.g., ZigBee, Z-Wave, LoRanWAN), Wi-Fi (IEEE 802.11 family of wireless communication), etc. It follows that the electronic control module 206 may be in a device separate from the support structure 204 such as, but not limited to, a separate WMD (not shown), a smart device (e.g., smart phone, tablet, personal computer, smart wearable, etc.). Accordingly, a variety of electronic control module implementations are contemplated within the scope of the present disclosure and not limited in this respect.

It should be appreciated that the electronic control module 206 may include a variety of sensors such as, but not limited to, photonic sensors (e.g., ambient light sensor), contact sensors including inductive sensors, electrical contact including capacitive and resistive, etc. Accordingly, a variety of sensors are contemplated within the scope of the present disclosure.

FIG. 3 illustrates utilization of a system for consistent attachment of a wearable medical device (WMD) in accordance with various alternative embodiments. In FIG. 3, a person 300 may be wearing a wearable medical device (WMD) 302 such as, but not limited to, a wearable cardioverter defibrillator (WCD). The example of the WMD 302 shown in FIG. 3 may include one or more parts such as, but not limited to, an electrode 304. As shown, the electrode 304 may be attached to a location on the skin of the person 300 proximate to the person's heart 306. The WMD 302 may be attached to a location on the skin of the person proximate to an area suitable for bearing the weight and bulk of the WMD 302 (e.g., the waist 308 of the person 300). A support structure 310 is shown coupled to the WMD 302. The support structure 310 may be coupled to the WMD 302 and go around the waist 308 of the person 300.

As shown in FIG. 3, the WMD may be proximate the waist 308 of the person 300. However, it is contemplated within the scope of the disclosure that the WMD 302 may be attached on the person 300 in any part of the body such as, but not limited to, the back, the arm, the thigh, the neck, chest, etc., and accordingly, the claimed subject matter is not limited in this respect.

It should be appreciated that the example shown in FIG. 3 may include a variety of configurations. For example, even though the WMD 302 may be shown to include the single electrode 304, the WMD 302 may include one or more electrodes based, at least in part, on the capabilities of the WMD 302. In the one example of FIG. 3, the WMD 302 may include an electrode surface (not shown) to facilitate an electrical connection between the WMD 302 and the electrode 304, where the electrical connection between the WMD 302 and the electrode 304 may facilitate shocking of the heart 306. In another example, the WMD 302 may include two electrodes attached close to the heart 306. Two electrodes may facilitate an electrical connection between the two electrodes to facilitate shocking of the heart 306. This example would allow the WMD 302 to be attached to the person 300 over a covering of the skin such as, but not limited to, a shirt, pants, sleeve, waistband, etc., and accordingly, the present disclosure is not limited in this respect.

The system, the WMD 302 and the support structure 310, may be configured as previously described with respect to FIGS. 1 and 2 to facilitate consistent attachment of the WMD 302 on the person 300 in accordance with various embodiments of the present disclosure.

After review of the present disclosure, it should be appreciated that it is contemplated within the scope and spirit of the present disclosure that the claimed subject matter may include a wide variety of support structures, WMDs, materials, configurations, etc. Accordingly, the claimed subject matter is not limited in these respects.

In some portions of the description, illustrative implementations of the disclosure may have been described with reference to the elements of the components described with respect to FIGS. 1-3. However, the described embodiments are not limited to these depictions. More specifically, some elements/components depicted in FIGS. 1-3 may be omitted from some implementations detailed herein. Furthermore, other elements not depicted in FIGS. 1-3 may be used to implement example apparatuses detailed herein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

What is claimed:

1. A system comprising:
a wearable medical device (WMD), the WMD including an electronics module having an adhesive layer and configured to be attachable to a person; and
a support structure configured to couple with the WMD, the support structure having a changeable length to facilitate consistent attachment of the WMD to the person.

2. The system of claim 1, wherein the support structure comprises a strap.

3. The system of claim 1, wherein the support structure comprises a mesh, the mesh configured to wrap around at least part of the WMD.

4. The system of claim 1 further comprising an electronic link between the WMD and the support structure.

5. The system of claim 4, wherein the electronic link comprises a data link.

6. The system of claim 1, wherein the electronics module includes an electronic control module, the electronic control module configured to facilitate control of the length of the support structure.

7. The system of claim 1, wherein the WMD comprises an adhesive electrode.

8. The system of claim 1, wherein the WMD comprises a wearable cardioverter defibrillator (WCD).

9. The system of claim 1, wherein the WMD comprises a WMD including an electrode, the electrode being attachable to the person.

10. The system of claim 1, wherein the support structure comprises elastane type material.

11. The system of claim 10, wherein the elastane type material comprises a polyether-polyurea copolymer.

12. The system of claim 1, wherein the support structure comprises a material made of nylon.

13. The system of claim 1, wherein the support structure comprises a material made of leather.

14. The system of claim 1, wherein the support structure comprises a material made from a combination of nylon and elastane.

15. The system of claim 1, wherein the support structure comprises a material made from cotton.

16. The system of claim 1, wherein the support structure comprises a material made from a combination of cotton and microfiber.

17. The system of claim 1, wherein the support structure comprises a material made from a combination of cotton, microfiber, nylon, and elastane.

18. The system of claim 1, wherein the support structure comprises smart textile.

19. The system of claim 18, wherein the smart textile is configured to react to stimuli from at least one of mechanical, thermal, chemical, electrical, or magnetic sources.

20. The system of claim 18, the smart textile is configured to react to environmental conditions from at least one of mechanical, thermal, chemical, electrical, or magnetic sources.

21. An apparatus comprising:
a support structure, the support structure configured to couple with a wearable medical device (WMD) having an electronics module with an adhesive layer and to have a changeable dimension; and
a dimension changeable control module configured to communicatively couple to the support structure, the dimension changeable control module configured to change a dimension of the support structure responsive to a received signal indicating an attachment issue from the WMD to maintain consistent attachment of the WMD to the person.
